(19) United States
(12) Patent Application Publication
(10) Pub. No.: US 2014/0212469 A1
(43) Pub. Date: Jul. 31, 2014

(54) SURFACE FUNCTIONAL BIOACTIVE GLASS SCAFFOLD FOR BONE REGENERATION

(71) Applicant: Missouri University of Science and Technology, Rolla, MO (US)

(72) Inventors: Mohamed N. Rahaman, Rolla, MO (US); Xin Liu, Rolla, MO (US); B. Sonny Bal, Rolla, MO (US); Yongxing Liu, Rolly, MO (US)

(73) Assignee: Missouri University of Science and Technology, Rolla, MO (US)

(21) Appl. No.: 14/166,559

(22) Filed: Jan. 28, 2014

Related U.S. Application Data
(60) Provisional application No. 61/849,512, filed on Jan. 28, 2013.

(51) Int. Cl. A61K 9/70

(52) U.S. Cl. CPC A61K 9/703
USPC 424/423; 424/602

(57) ABSTRACT

A bioactive glass scaffold treated to modify the surface of the bioactive glass scaffold for regenerating new bone tissue is disclosed. In some embodiments, the bioactive glass scaffold may include pores in a grid-like structure to promote the ingrowth of bone tissue, and the modified surface layer may include a hydroxyapatite-like surface. The bioactive glass scaffold is inexpensive and easy to fabricate and regenerates new bone faster than the existing synthetic implants. The bioactive glass scaffold may be loaded with a biomolecule, such as BMP-2, for delivery to the implantation site.

### CROSS REFERENCE TO RELATED APPLICATION

This application is a non-provisional that claims benefit to U.S. Provisional Patent Application No. 61/849,512, filed on Jan. 28, 2013, which is herein incorporated by reference in its entirety.

### GOVERNMENTAL SUPPORT

This invention was made with Government support under Grant No. R15AR056119-01 awarded by the National Institutes of Health. The Government has certain rights in the invention.

### FIELD

The present document relates to bioactive glass scaffolds to be used to regenerate large bone defects in mammals, and in particular to bioactive glass scaffolds having a modified surface area for enhancing bone repair and regeneration.

### BACKGROUND

There is a clinical need for synthetic scaffolds that can be used to regenerate large bone defects in mammals which result from trauma, malignancy, and congenital diseases. Autologous bone grafts (taken from the patient) are the gold standard for treatment and bone allografts (taken from a cadaver) are alternatives to autologous bone grafts. However, both kinds of bone grafts suffer from limitations such as donor site morbidity, limited supply (autografts), and possible transmission of diseases and high cost (allografts). Synthetic scaffolds have been gaining interest as an alternative to bone grafts. However, the existing synthetic scaffolds are generally expensive, hard to manufacture, and suffer from slow bone regeneration. Furthermore, currently available synthetic bone graft substitutes have low strength and are limited to the repair of non-loaded bone only and cannot be used to replace structural bone loss.

Therefore, there is a need to provide new and improved synthetic scaffolds that include bioactive glass, which are inexpensive, easy to fabricate, and when treated to modify the surface, regenerate new bone tissue faster than the existing synthetic scaffolds. Additionally, there is a great need for porous biocompatible scaffolds that can replicate the structure and function of bone and have the requisite mechanical properties for reliable long-term cyclical loading during weight bearing.

### SUMMARY OF INVENTION

In one embodiment, a bioactive glass scaffold is provided to enhance new bone formation including a bioactive glass material formed in a three-dimensional macroporous grid-like microstructure and having a modified surface layer on the surface of the bioactive glass scaffold. The modified surface layer increases the surface area of the bioactive glass, and at least part of the modified surface layer is converted to a calcium phosphate material.

In another embodiment, a method of manufacturing a bioactive glass scaffold is provided that includes: grinding a bioactive glass into fine particles; mixing the fine particles of bioactive glass with a processing aid and a liquid to form a slurry; fabricating a three-dimensional macroporous grid-like microstructure of the slurry to form a bioactive glass scaffold; and modifying the surface of the bioactive glass scaffold with a glass modifier. The modified surface layer increases the surface area of the bioactive glass scaffold to enhance new bone formation.

### BRIEF DESCRIPTION OF DRAWINGS

FIG. 1A is an optical image of as-fabricated bioactive glass scaffolds prepared by robocasting;

FIG. 1B is a higher magnification SEM image of the bioactive glass scaffold showing the glass struts (filaments) and pores with the bioactive glass scaffolds having a porosity of ˜50%; strut diameter of ˜300 μm, and a pore width of ˜300 μm;

FIGS. 2A-2D are SEM images of (A) the as-fabricated bioactive glass scaffold (included for comparison) and (B) a bioactive glass scaffold after reaction for 6 days in 0.25M K₂HPO₄ solution (60° C.; pH=12.0); (C) the surface of the reacted layer (higher magnification) for a bioactive glass scaffold showing a porous structure of fine calcium phosphate particles; (D) a cross-sectional view of a bioactive glass strut showing the thickness of the reacted layer;

FIGS. 3A-3C are SEM images of the surface of the bioactive glass scaffolds reacted (A) 1 day; (B) 3 days; and (C) 6 days in aqueous phosphate solution, showing differences in morphology as a function of reaction time;

FIG. 4 is a graph showing X-ray diffraction patterns of the as-fabricated bioactive glass (13-93) scaffolds and after reaction for 1, 3, and 6 days in 0.25 M K₂HPO₄ solution (60° C.; pH=12.0);

FIGS. 5A-5E are Von Kossa stained images of bioactive glass (13-93) scaffolds after 6 weeks in rat calvarial critical sized defects (4.6 mm in diameter), wherein the dark stain is sensitive to phosphate material (e.g., bone and converted layer of bioactive glass). (A) as-fabricated bioactive glass scaffold; (B) surface modified bioactive glass scaffold reacted for 3 days in aqueous phosphate solution; (C)-(E) surface modified bioactive glass scaffold reacted for 1, 3, and 6 days in aqueous phosphate solution and loaded with BMP-2 (1 μg/defect). (G denotes glass from the scaffold);

FIG. 6 is a graph showing the amount of BMP-2 released from the as-fabricated scaffold (0 d) and the scaffolds pretreated for 1 day, 3 days and 6 days at different time periods in a solution composed of a mixture of FBS and PBS. Each scaffold was loaded with 1 μg of BMP-2;

FIG. 7 is a graph showing the cumulative amount of BMP-2 released from the scaffolds, as a fraction of the amount of BMP-2 initially loaded into the scaffolds, as a function of time in the FBS/PBS solution;

FIGS. 8A-8D are von Kossa stained sections (A1-D1) and H&E stained sections (A2-D3) of rat calvarial defects implanted with bioactive scaffolds as fabricated (0 d) and pretreated for 1 day, 3 days, and 6 days in aqueous phosphate solution at 6 weeks post implantation; Scale bar=1 mm for (A1-D2) and 200 μm for (A3-D3). G=bioactive glass; NB=new bone; O=old bone; arrows indicate blood vessels, arrowheads indicate the edges of old bone;

FIGS. 9A-9C are von Kossa stained sections (A1-C1) and H&E stained sections (A2-C3) of rat calvarial defects implanted with bioactive glass scaffolds pretreated for 1 day, 3 days, and 6 days in aqueous phosphate solution and loaded with BMP-2 (1 μg/defect) at 6 weeks post implantation. Scale bar=1 mm for (A1-C2) and 200 μm for (A3-C3). G=bioactive glass; NB=new bone; O=old bone; M=bone marrow-like tissue, arrowheads indicate the edges of old bone;

FIG. 10 is a graph showing the percent new bone formed in rat calvarial defects implanted with bioactive glass scaffolds of 13-93 glass at 6 weeks post implantation: as fabricated (0 d); pretreated for 1 day, 3 days, and 6 days in aqueous phosphate solution; pretreated and loaded with BMP-2 (1 μg/defect). The new bone formed was determined as a percent of the available pore space in the scaffolds. (*significant difference when compared to 0 d; **significant difference when compared to 1 d, 1 d, and 6 d; p<0.05);

FIG. 11 is a graph showing the percent bone marrow-like tissue formed in rat calvarial defects implanted with scaffolds of 13-93 glass at 6 weeks post implantation: as fabricated (0 d); pretreated for 1 day, 3 days, and 6 days in aqueous phosphate solution; pretreated and loaded with BMP-2 (1 μg/defect). (*significant difference when compared to 0 d; **significant difference when compared to 0 d, 1 d, 3 d and 6 d; p<0.05);

FIG. 12 is a graph showing the percent fibrous tissue formed in rat calvarial defects implanted with scaffolds of 13-93 glass at 6 weeks post implantation: as fabricated (0 d); pretreated for 1 day, 3 days, and 6 days in aqueous phosphate solution; pretreated and loaded with BMP-2 (1 μg/defect). (*significant difference when compared to 0 d; **significant difference when compared to 0 d, 1 d, 3 d, and 6 d; p<0.05);

FIGS. 13A-13F are back-scattered SEM images of rat calvarial defects implanted with bioactive glass scaffolds at 6 weeks post implantation: (A), (B) as-fabricated scaffolds; (C), (D) bioactive glass scaffolds pretreated for 3 days in aqueous phosphate solution; (E), (F) bioactive glass scaffolds pretreated for 3 days in aqueous phosphate solution and loaded with BMP-2. (NB=new bone; G=bioactive glass).

### DETAILED DESCRIPTION

As described herein, embodiments of a bioactive glass scaffold having a bioactive glass and a modified surface layer for enhancing bone repair and regeneration. The bioactive glass scaffold may be a porous three-dimensional (3D) scaffold for implantation and promotion of bone growth. In addition, a method of manufacturing the bioactive glass scaffold is disclosed which includes forming a grid-like microstructure of bioactive glass and modifying the surface of the glass scaffold by a chemical treatment to enhance new bone formation in mammals. The bioactive glass scaffold can be implanted into a subject to promote bone growth. The bioactive glass scaffold may further include biomolecules such as growth factors which may be incorporated in the modified surface layer. In some embodiments, the modified surface layer of the bioactive glass scaffold may include osteoinductive growth factors such as bone morphogenetic protein (BMP) to stimulate bone formation.

Embodiments of the bioactive glass scaffold provide several advantages. First, the bioactive glass scaffolds may provide a low-cost alternative to bone autografts and allografts (the current treatment options); second, the bioactive glass scaffolds convert to hydroxyapatite or a hydroxyapatite-like material, the mineral constituent of bone, so there is no need to remove the bioactive glass scaffold from the body in a later surgery, as in the case for some synthetic bone graft substitutes; and third, the bioactive glass scaffolds exhibit superior strength to other synthetic bone graft substitutes, so such scaffolds can be applied to the regeneration of load-bearing or load-sharing bones, as well as non-loaded bone.

The bioactive glass scaffold includes a bioactive glass as the foundation for the scaffold. In some embodiments, the bioactive glass is preferably a silicate bioactive glass. Alternatively, some embodiments of the bioactive glass can also include one or more glasses from other glass-forming systems, such as borate, phosphate, borosilicate, etc. In one embodiment, the bioactive glass may be a silicate bioactive glass with a composition designated as 13-93 (6Na₂O, 12K₂O, 5MgO, 20CaO, 4P₂O₅, 53SiO₂, wt. %). In another embodiment, the bioactive glass scaffold may be B₂O₃-doped silicate bioactive glass scaffolds with a fibrous microstructure.

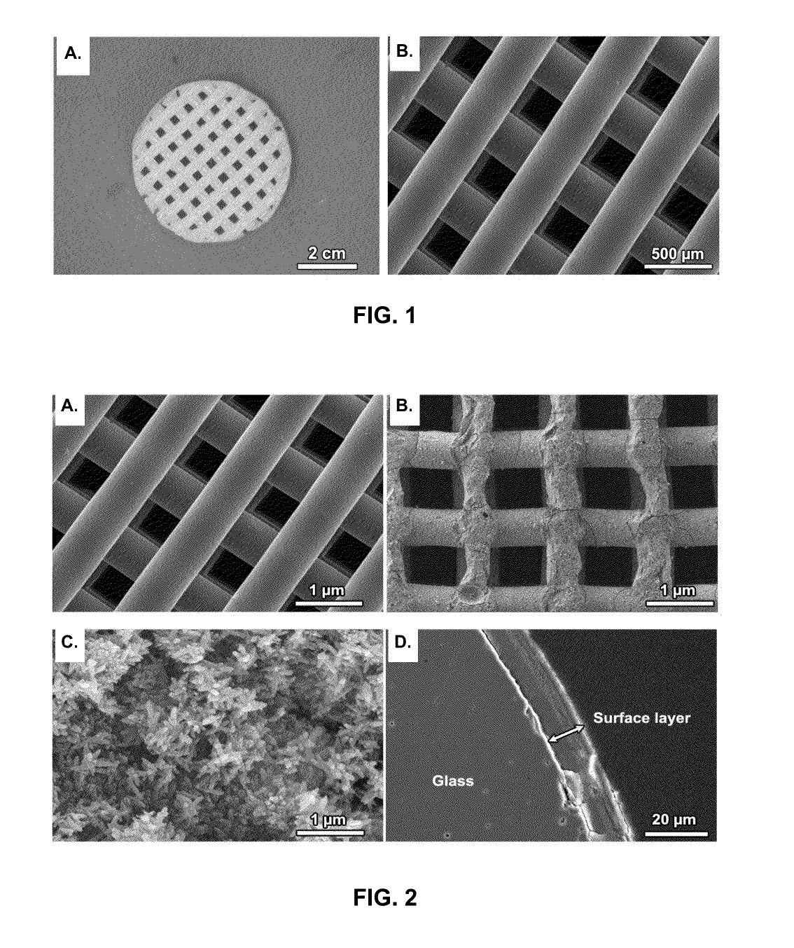

Embodiments of the bioactive glass scaffold are macroporous with a high-surface-area modified surface layer formed in a grid-like microstructure. FIG. 1 illustrates various embodiments of bioactive glass scaffolds with grid-like microstructures. In various embodiments, the bioactive glass scaffold may include interconnected pores with diameters ranging from about 100 μm to about 300 μm, about 200 μm to about 400 μm, and about 300 μm to about 500 μm. Interconnected pores of about 100 μm are recognized as the minimum requirement for supporting tissue ingrowth, but pores of about 300 μm or larger may be required for enhanced bone ingrowth and capillary formation. Furthermore, the porosity of the bioactive glass scaffold may range from about 20% to about 80%. In various embodiments, the porosity of the bioactive glass may be about 50%. In a preferred embodiment, the porosity of the bioactive glass scaffold may be about 47%±1%. The filaments in the bioactive glass scaffold may have a width from about 50 μm to about 500 μm. In an embodiment, the filament width may be about 330 μm.

In some embodiments, the bioactive glass scaffold may be pretreated with a glass modifier to create a modified surface layer. The modified surface layer of the bioactive glass scaffold may create fine pores (nanometers to a few microns in size) to modify the surface roughness and increase the surface area of the bioactive glass scaffolds. The glass modifiers of the bioactive glass may include but are not limited to one or more elements from the alkali metals (e.g., Li, Na, K, etc), alkali-earth metals (Ca, Mg, etc), an aqueous phosphate solution, and other elements from the periodic table. The surface area of the modified surface layer may range from about 1 m²/g to about 100 m²/g.

Once treated with the glass modifier, the modified surface layer may be converted to include a calcium phosphate material. In an embodiment, the calcium phosphate material may be hydroxyapatite (HA) or a hydroxyapatite-like material. Once the bioactive glass scaffold is implanted, the calcium phosphate or HA-like material may further crystallize to HA. In an embodiment, the HA-like material on the modified surface layer may be formed by reaction of the bioactive glass scaffold in an aqueous phosphate solution. HA has been shown to improve the capacity of borate and silicate bioactive glass to support cell proliferation and differentiation in vitro. A rough modified surface layer of carbonated HA may improve the capacity of the bioactive glass scaffolds to support cell proliferation in vitro and to enhance bone formation in vivo.

In addition to material composition and microstructure, scaffold healing to bone in vivo can be markedly affected by other variables, such as the modified surface layer composition and structure, release of osteoinductive growth factors, and presence (or absence) of living cells. A bioactive glass scaffold with a modified surface layer may be implanted as prepared, or in combination with biomolecules, for the promotion of bone growth and regeneration.

In some embodiments, the surface modified bioactive glass scaffold may incorporate biomolecules, such as growth factors, drugs, antibodies, antibiotics or other molecules. The biomolecules may be adsorbed on the porous surface of the surface modified layer of the bioactive glass scaffold or the biomolecules can be chemically bonded onto the surface modified layer. In an embodiment, the modified surface layer of the bioactive glass scaffold may further be loaded with BMP-2. The bioactive glass scaffold may be loaded with about 1 μg/scaffold in one embodiment.

Bioactive glass scaffolds with silicate 13-93 may have promising mechanical properties for the repair of loaded bone. Pretreatment of the bioactive glass scaffolds in an aqueous phosphate solution to convert a modified surface layer to HA or HA-like material, or loading the pretreated bioactive glass scaffolds with BMP-2, may enhance the capacity of the bioactive glass scaffolds to regenerate bone in an osseous defect. The bioactive glass scaffolds pretreated with a glass modifier or BMP-loaded may support faster bone regeneration. Pretreatment of bioactive glass scaffolds to form an HA-like modified surface layer prior to implantation may provide a new and effective approach for regenerating bone in osseous defects. The modified surface layer can enhance the osteoconductivity of the bioactive glass scaffolds and provide a substrate for delivery of therapeutics such as growth factors.

Further provided herein is a method of fabricating the bioactive glass scaffold. The method may include the steps of 1) fabricating a porous bioactive glass scaffold in a desired 3D shape with bioactive glass, and 2) surface modifying the porous 3D bioactive glass scaffold. In some embodiments, the starting material includes a silicate bioactive glass, such as 13-93 glass. The bioactive glass may be a commercially available glass. The bioactive glass may be ground into fine particles (about 1-2 μm) using conventional materials processing methods which then may be combined with a liquid to form a slurry. In an embodiment, the liquid may be water. In some embodiments, the slurry may be composed of about 62.5 wt % glass particles, about 7.5 wt % Pluronic® F-127 (used as a processing aid), and 30 wt % distilled water.

In some embodiments, the bioactive glass scaffold may be formed from the slurry using a solid freeform fabrication method (also known as rapid prototyping), such as robocasting. In robocasting, the bioactive glass scaffold is formed layer-by-layer using a computer-aided design (CAD) file in the fabricating step. However, other methods used for forming glass and ceramics can be used, such as sintering of particles or fibers, sol-gel processing, polymer foam replication, and freezing of suspensions.

FIG. 1 illustrates various embodiments of bioactive glass scaffolds with grid-like microstructures. In an embodiment, the bioactive glass scaffold may be prepared with a grid-like microstructure using robocasting. The slurry may be extruded through a syringe tip having a diameter of about 410 μm with the center-to-center spacing between adjacent filaments of about 910 μm. A post-forming thermal process may be used to dry the bioactive glass scaffolds, decompose the processing aid used in the robocasting process, and then bond the glass particles into a dense network. In one embodiment, the bioactive glass scaffolds may be heated slowly (about 0.5° C./min with a few isothermal holds) to about 600° C. in flowing O₂ gas to decompose the processing aid and sinter in air for about 1 hour at about 700° C. (heating rate of about 5° C./min) to densify the glass filaments. The bioactive glass scaffolds may further be heated at 250° C. for about 12 hours to sterilize the bioactive glass scaffolds.

In the surface modifying step, the bioactive glass scaffold may be pretreated with a glass modifier. The glass modifier of the bioactive glass may include but is not limited to one or more elements from the alkali metals (e.g., Li, Na, K, etc), alkali-earth metals (Ca, Mg, etc), an aqueous phosphate solution, and other elements from the periodic table. In an embodiment, the glass modifier may be an aqueous phosphate solution treated under specified conditions. Alternatively, aqueous solutions without phosphate ions, or aqueous solutions containing ions or molecules in addition to phosphate ions can also be used.

In some embodiments, the bioactive glass scaffold may be pretreated with a glass modifier for about 1 to about 6 days. In one embodiment, the bioactive glass scaffold may be pretreated with a glass modifier for about 3 days. In various embodiments, the bioactive glass scaffold may be pretreated with a glass modifier at various times in an aqueous phosphate solution to create the modified surface layer on the bioactive glass scaffold. In an embodiment, the modified surface layer may be converted to calcium phosphate, hydroxyapatite (HA), or an HA-like material. The pretreatment may form a modified surface layer of HA on the surface of the bioactive glass scaffold.

In some embodiments, the thickness of the modified surface layer may be controlled by the time and temperature of the pretreatment of the bioactive glass scaffold with the glass modifier. The modified surface layer thickness may range from about 1 μm to about 20 μm. In various embodiments, the modified surface layer thickness may range from about 1 μm to about 10 μm, about 5 μm to about 15 μm, and about 10 μm to about 20 μm. In an embodiment, the modified surface layer may be about 5 μm thick. The thickness of the modified surface layer may be measured using Image J software. Table I shows the thickness of the modified surface layer on silicate (13-93) bioactive glass scaffolds with increasing reaction time in a 0.25M aqueous phosphate solution from 1-6 days at 60° C. and pH=12.0.

TABLE I

| Immersion | Thickness | Surface area |
|---|---|---|
| time (d) | (μm) | (m²/g) |
| 1 | 2 ± 1 | 19 ± 2 |
| 3 | 5 ± 2 | 30 ± 3 |
| 6 | 13 ± 2 | 47 ± 1 |

The surface modified bioactive glass scaffold can be used as prepared, or after incorporation of biomolecules or other species in the modified surface layer of the bioactive glass scaffold, such as growth factors, drugs, antibodies, antibiotics or other molecules. The biomolecules can be adsorbed on the porous surface of the surface modified layer of the bioactive glass scaffold or they can be chemically bonded onto the surface modified layer. In an embodiment, the modified surface layer of the bioactive glass scaffold may further be loaded with BMP-2. The bioactive glass scaffold may be loaded with about 1 μg/scaffold in one embodiment.

As-fabricated glass scaffolds prepared by sintering melt-derived glass particles may have a dense smooth surface, as seen in FIG. 1, while the glass phase in bioactive glass scaffolds prepared by techniques such as sol-gel processing may contain fine surface pores. Upon implantation in vivo, the bioactive glass reacts with the body fluid and converts to a porous HA-like material. The conversion reaction starts at the surface and moves inward. In the case of silicate bioactive glass scaffolds (such as 13-93 and 45S5), it is widely accepted that the conversion takes place in stages. The conversion of the modified surface layer starts with the formation of a silica-rich layer on the surface of the glass by ion-exchange reactions, which is followed by deposition of an amorphous calcium phosphate (ACP) material on the silica-rich layer. As the ACP layer grows with time, crystallization to HA takes place.

Pretreatment of silicate 13-93 bioactive glass scaffolds prior to implantation with a glass modifier leads to the formation of a nanostructured calcium phosphate that is known to be favorable for protein adsorption and early bone formation. Conversion of bioactive glass in an aqueous phosphate solution forms a calcium phosphate modified surface layer which enhances adhesion, proliferation and differentiation of osteoblast cells in vitro.

The bioactive glass scaffold may be implanted into or onto bone to stimulate bone growth. Without being limited to a particular theory, bone growth may be stimulated by the delivery of a plurality of biomolecules, the modified surface layer including HA, or ingrowth of cells into the pores of the bioactive glass scaffold. New bone formation in the bioactive glass scaffolds may be evaluated using histomorphometric techniques and scanning electron microscopy. The bioactive glass scaffold may remain implanted as it becomes incorporated into the bone. In addition, the bioactive glass scaffold with a modified surface layer may be converted to HA at a slower rate than a scaffold without pretreatment or modified surface layer. Without being limited to a particular theory, the slow conversion to HA may reduce local increases in pH and the concentration of ions released from the glass, which may help cell proliferation around the bioactive glass scaffold.

The greater capacity of the grid-like microstructure to support bone infiltration may result from the uniform microstructure of interconnected pores with a favorable size. As described earlier, interconnected pores of size about 100 μm are recognized as the minimum requirement for supporting tissue ingrowth, but pores of about 300 μm or larger may be required for enhanced bone ingrowth and capillary formation. In the grid-like microstructure, the pores are all interconnected, have the same size, and are not constricted at the necks between adjacent pores. In comparison, the pores in oriented scaffolds have a smaller pore diameter and limited interconnectivity between adjacent pores than the grid-like microstructure of the bioactive glass scaffold. In an embodiment, new bone formation may be osteoconductive and may infiltrate the pores of the bioactive glass scaffold along the edge of the bioactive glass scaffold.

### EXAMPLES

### Example 1

### Preparation of Bioactive Glass (13-93) Scaffolds

FIG. 1 illustrates exemplary bioactive glass scaffolds with grid-like microstructures. Bioactive glass scaffolds of silicate 13-93 glass (composition 53SiO₂, 6Na₂O, 12K₂O, 5MgO, 20CaO, 4P₂O₅; wt %) with a grid-like microstructure were prepared using a robotic deposition (robocasting) method. Briefly, a slurry was prepared by mixing 40 vol % glass particles (about 1 μm) with a 20 wt % aqueous Pluronic® F-127 solution in a planetary centrifugal mixer (ARE-310, THINKY U.S.A. Inc, Laguna Hills, Calif., USA). Then the slurry was loaded into a robotic deposition device (RoboCAD 3.0, 3-D Inks, Stillwater, Okla.) and extruded through a syringe (tip diameter=410 μm) onto an Al₂O₃ substrate to form a 3D bioactive glass scaffold. The extruded filaments were deposited at right angles to the filaments in the adjacent layer, with a center-to-center spacing between the filaments of 910 μm in the plane of deposition. After forming, the bioactive glass scaffolds were dried for 24 h at room temperature, heated slowly (0.5° C./min with a few isothermal holds) to 600° C. in flowing oxygen to burn out the polymer aids, and sintered in air for 1 h at 700° C. (heating rate=5° C./min) to densify the glass filaments. The as-fabricated constructs were sectioned and ground into thin discs (4.6 mm in diameter×1.5 mm), washed twice with deionized water and twice with ethanol, dried in air, and then sterilized by heating for 12 h at 250° C. The bioactive glass scaffolds had a porosity of about 50%, a filament width of about 330 μm, and a pre width of about 300 μm.

### Example 2

### Surface Modification of Bioactive Glass Scaffolds

The as-fabricated scaffolds were modified prior to implantation by reacting them in an aqueous phosphate solution to convert a surface layer of the bioactive glass to a hydroxyapatite (HA)-like modified surface layer. In the surface modification process, the bioactive glass scaffolds were immersed for 1, 3, and 6 days in 0.25 M K₂HPO₄ solution at 60° C. and a starting pH=12.0 (obtained by adding the requisite amount 2M NaOH solution). The mass of the bioactive glass scaffolds to the volume of the K₂HPO₄ solution was kept constant at 1 g per 200 ml, and the system was stirred gently each day. These reaction conditions were based on previous studies on the conversion of bioactive glasses to HA. After each reaction time, the bioactive glass scaffolds were removed from the solution, washed twice with deionized water, and twice with anhydrous ethanol to displace residual water from the bioactive glass scaffolds. The bioactive glass scaffolds were removed from the ethanol, dried for at least 24 h at room temperature, and stored in desiccator.

In this reaction process, a surface layer of the bioactive glass was converted to a modified surface layer composed of a calcium phosphate material. The thickness and the crystallinity of the modified surface layer were controlled by the time and temperature of the reaction. After the surface treatment, the bioactive glass scaffolds may be washed twice with distilled water and then twice with ethanol, and dried at room temperature for one day.

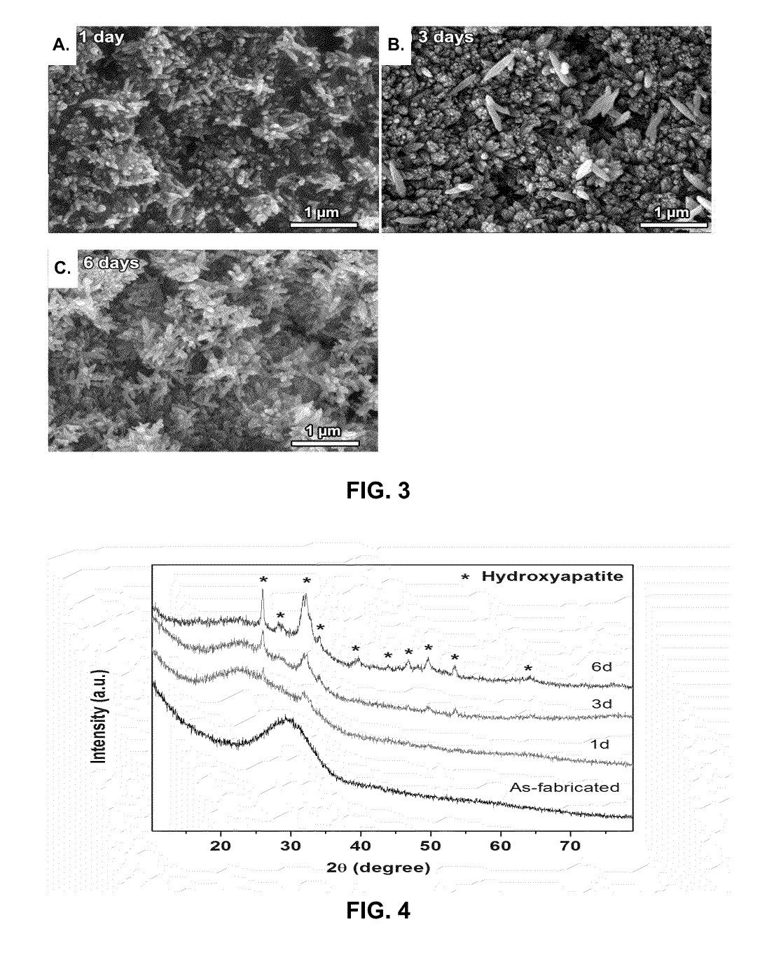

The morphology, crystallinity, specific surface area, and thickness of the converted layer of the bioactive glass scaffolds changed with an increase in the reaction time (1-6 days) in the K₂HPO₄ solution (FIGS. 2 and 3; Table I). While the converted layer was predominantly amorphous with a small amount of needle-like HA crystals after a reaction time of 1 day, the amount of needle-like HA crystals increased with reaction time and, after 6 days, the modified surface layer consisted predominantly of needle-like HA crystals.

Table I shows the thickness and specific surface area of the modified surface layer formed by reacting silicate (13-93) bioactive glass scaffolds for the times shown in 0.25M K₂HPO₄ solution at 60° C. and starting pH=12.0.

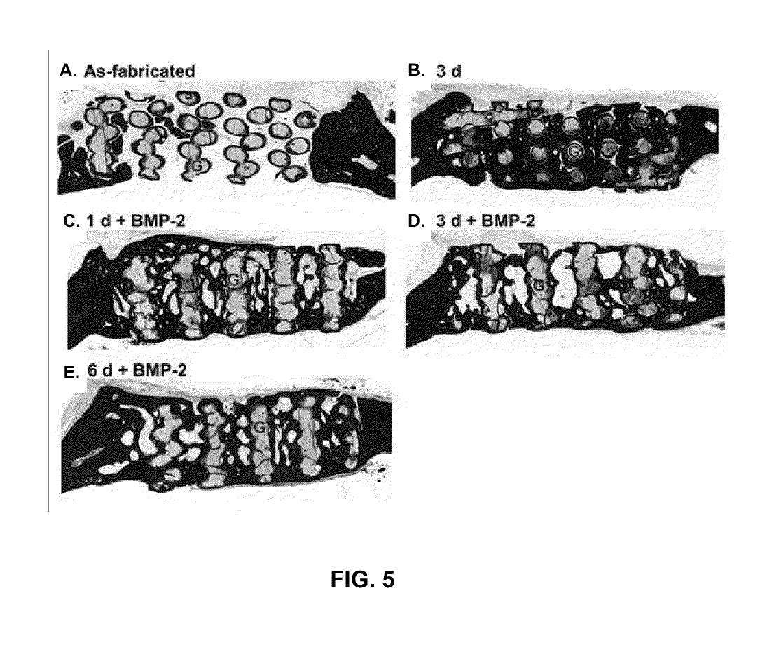

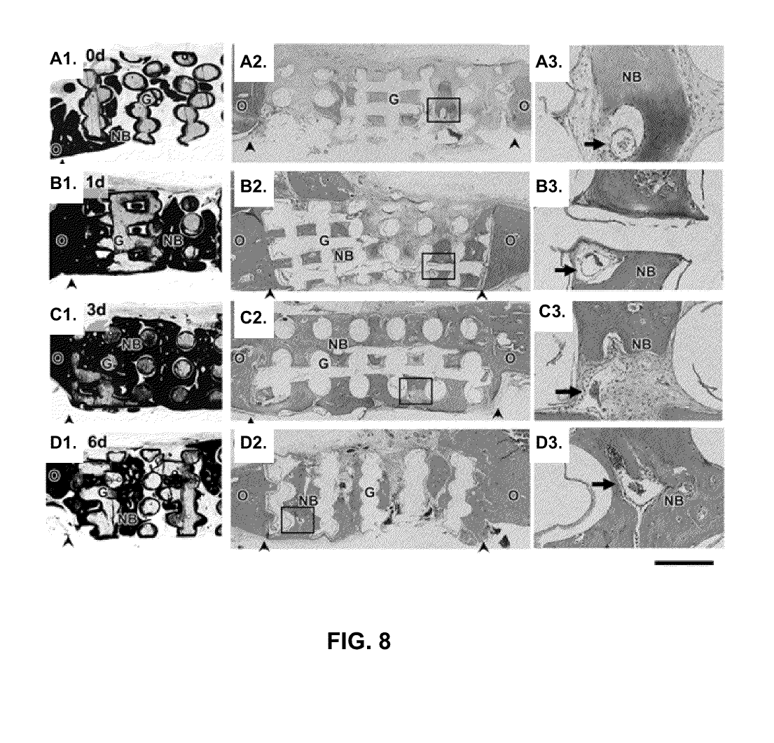

The osteoconductivity and osteoinductivity of calcium phosphate and bioactive glass scaffolds for bone repair varied with the material characteristics, such as chemical composition, specific surface area, and micrometer size pores (0.5-10 μm) in the bioactive glass scaffold. While the scaffolds pretreated for 1, 3, and 6 days were all effective, the bioactive glass scaffold pretreated for 3 days showed a significantly better capacity to enhance new bone formation in vivo (FIGS. 5 and 8). The modified surface layer of that bioactive glass scaffold (pretreated for 3 days) had a combination of characteristics such as morphology, crystallinity and specific surface area which was intermediate between those for the modified surface layer formed by pretreatment for 1 and 6 days.

### Example 3

### Characterization of Modified Surface Layer

The modified surface layer of the bioactive glass scaffolds was sputter-coated with Au/Pd and examined in a scanning electron microscope, SEM (S-4700; Hitachi, Tokyo, Japan), using an accelerating voltage of 15 kV and a working distance of 8 mm. Some bioactive glass scaffolds were also mounted in epoxy resin, sectioned, polished to expose the cross-sections of the glass filaments, and examined in the SEM (S-4700; Hitachi). The thickness of the modified surface layer was determined from more than 15 measurements in the SEM images using the ImageJ software (National Institutes of Health, USA), and expressed as a mean value±standard deviation (sd).

The modified surface layer was removed by vigorously shaking the bioactive glass scaffolds and used in determining its surface area and phase composition. Surface area measurements were made using nitrogen gas adsorption (Nova 2000e; Quantachrome, Boynton Beach, Fla., USA). The volume of nitrogen adsorbed and desorbed at different gas pressures was measured and used to construct adsorption-desorption isotherms. Eleven points of the adsorption isotherm, which initially followed a linear trend implying monolayer formation of adsorbate, were fitted by the Brunauer-Emmett-Teller equation to determine the surface area.

The presence of crystalline phases in the modified surface layer was determined using X-ray diffraction (XRD) (D/mas 2550 v; Rigaku, The Woodlands, Tex., USA). The material was ground into a powder and analyzed using Cu K_{α} radiation (A=0.15406 nm) at a scan rate of 1.8°/min in the 28 range 10-80°.

Referring to FIGS. 2A and 2B, when compared to the as-fabricated scaffold, the modified surface layer of the bioactive glass scaffold became rougher after reaction in the phosphate solution, indicating that the surface of the bioactive glass scaffold was modified by the reaction. High magnification images showed that the modified surface layer was composed of a porous structure of fine particles (FIG. 2C); examination of the cross sections of the struts (filaments) of the bioactive glass scaffold showed the thickness of the modified surface layer (FIG. 2D). The thickness of the modified surface layer was measured using Image J software and the values are given in Table I. With increasing reaction time in the phosphate solution from 1 day to 6 days, the thickness of the reacted layer increased from about 2 μm to about 13 μm, and the surface area increased from about 20 m²/g to about 50 m²/g.

FIG. 3 shows SEM images of the modified surface layer of the bioactive glass scaffolds after reaction of the bioactive glass scaffolds in aqueous phosphate solution for 1 day, 3 days, and 6 days. A change in the morphology of the surface layer was seen with different reaction times.

The pretreated bioactive glass scaffolds showed a much slower conversion rate to HA in vivo than the as-fabricated scaffold (no pretreatment). After the six-week implantation, the thickness of the converted layer in the pretreated bioactive glass scaffolds showed little change, whereas the converted layer of the as-fabricated scaffolds had a thickness about 20 μm. When compared to the as-fabricated scaffolds, the slow conversion of the pretreated bioactive glass scaffolds in vivo had the effect of reducing local increases in pH and the concentration of ions released from the glass (e.g., Na⁺; K⁺), particularly soon after implantation when the conversion rate of the as-fabricated scaffolds is fast. Large increases in the local pH are detrimental to cell proliferation.

### Example 4

### Loading Pretreated Bioactive Glass Scaffolds with Bone Morphogenetic Protein-2 (BMP-2)

Some of the pretreated bioactive glass scaffolds described above were loaded with bone morphogenetic protein-2 (BMP-2) prior to implantation. In the process, a solution of BMP-2 (Shenandoah Biotechnology Inc., PA, USA) in citric acid was prepared by dissolving 10 μg of BMP-2 in 100 μl sterile citric acid (pH=3.0). Then 10 μl of the BMP-2 solution was pipetted on to each bioactive glass scaffold (4.6 mm in diameter×1.5 mm). The amount of BMP-2 loaded into the bioactive glass scaffolds was equivalent to 1 μg per bone defect (or per scaffold) in the animal model. The BMP-loaded bioactive glass scaffolds were kept for about 24 h in a refrigerator at 4° C. to dry them prior to implantation. For comparison, the as-fabricated scaffolds (no pretreatment in the phosphate solution) were also loaded with BMP-2 using the same procedure.

### Example 5

### Release Profile of Bone Morphogenetic Protein-2 (BMP-2) from Pretreated Bioactive Glass Scaffolds

The release of BMP-2 from the bioactive glass scaffolds into a medium composed of equal volumes of fetal bovine serum (FBS) and phosphate-buffered saline (PBS) plus 1 vol % penicillin was measured as a function of time in vitro. Each bioactive glass scaffold was immersed in 500 μl of the solution in a 2.0 ml microtube and kept at 37° C. in an incubator. Three replicates were used for each group at each time point. At selected times (1 h, 8 h, 1 d, 3 d, 7 d, 14 d), the solution was completely removed and replaced with fresh solution. The amount of BMP-2 released into the solution was measured using by an enzyme-linked immunosorbent assay (ELISA) kit (Pepro Tech, Rocky Hill, N.J., USA) according to the manufacturer's instructions.

### Example 6

### Animals and Surgical Procedure

All animal experimental procedures were approved by the Animal Care and Use Committee, Missouri University of Science and Technology, in compliance with the NIH Guide for Care and House of Laboratory Animals (1985). Seven groups of bioactive glass scaffolds, described in Table II, were implanted in rat calvarial defects for 6 weeks. The bioactive glass scaffolds were assigned randomly, but bioactive glass scaffolds with and without BMP-2 were not mixed in the same animal. The implantation time was used because our previous studies had shown considerable bone regeneration in bioactive glass scaffolds composed of BMP-loaded hollow HA microspheres after implantation for the same time in rat calvarial defects.

TABLE II

| | Pretreatment | | |
|---|---|---|---|
| | time in K₂HPO₄ | BMP-2 | Number of |
| Scaffold group | solution (day) | loading | Defects |
| As fabricated (0 d) | — | — | 10 |
| 1 d | 1 | — | 5 |
| 3 d | 3 | — | 10 |
| 6 d | 6 | — | 5 |
| 1 d + BMP | 1 | 1 μg/defect | 5 |
| 3 d + BMP | 3 | 1 μg/defect | 5 |
| 6 d + BMP | 6 | 1 μg/defect | 5 |

Thirty male Sprague Dawley rats (3 months old; weight=350-400 g, Harlan Laboratories Inc., USA) were maintained in the animal facility for 2 weeks to become acclimated to diet, water and housing. The rats were anesthetized with a combination of ketamine (72 mg/kg) and xylazine (6 mg/kg) and maintained under anesthesia with ether gas in oxygen. The surgical site was shaved, scrubbed with iodine and draped. Using sterile instruments and aseptic technique, a full-thickness defect (4.6 mm in diameter) was created in the central area of each parietal bone using a saline-cooled trephine drill. The dura mater was not disturbed. The bilateral defects were randomly implanted with 5 or 10 bioactive glass scaffolds per group. The periosteum and skin were repositioned and closed using wound clips. All animals were given a dose of ketoprofen (3 mg/kg) intramuscularly and about 200 μl penicillin subcutaneously post-surgery. The animals were monitored daily for condition of the surgical wound, food intake, activity and clinical signs of infection. After 6 weeks, the animals were sacrificed by CO₂ inhalation, and the calvarial defect sites with surrounding bone and soft tissue were harvested for subsequent evaluation.

The amount of new bone formed in the as-fabricated 13-93 bioactive glass scaffolds, determined as a fraction of the available pore area, was 32±13% after the six-week implantation. In comparison, the amount of new bone formed in 13-93 bioactive glass scaffolds with an oriented microstructure (porosity=50%; pore diameter=50-100 μm) was 37±8% after implantation for 12 weeks in the same osseous defect model (rat calvarial model) (Table III). Table III shows the comparison of new bone formed in bioactive glass scaffolds composed of silicate 13-93 glass with different microstructures after implantation in rat calvarial defects (4.0-4.6 mm in diameter □ 1.5 mm). The amount of new bone is shown as a percent of the available pore space in the bioactive glass scaffolds.

TABLE III

| Microstructure | Porosity | Pore size | New bone | Implantation |
|---|---|---|---|---|
| of scaffolds | (%) | (μm) | (%) | time (weeks) |
| Grid-like | 47 | 300 × 300 × 150 | 32 ± 13 | 6 |
| Trabecular | 80 | 100-500 | 25 ± 12 | 12 |
| Oriented | 50 | 100-150 | 37 ± 8 | 12 |
| Fibrous | 50 | 50-500 | ~17* | 12 |

*Estimated from 8.5% based on the total defect area

For 13-93 bioactive glass scaffolds with a trabecular microstructure (similar to dry human trabecular bone) (porosity=80%; pore size=100-500 μm), only 25±12% of the available porosity was infiltrated with new bone after an implantation time of 12 weeks in the same osseous defect model. Bioactive glass scaffolds of 13-93 glass with a fibrous microstructure of thermally bonded short fibers (porosity=50%; pore size=50-500 μm), showed new bone formation equal to 8.5% of the total defect size when implanted in rat calvarial defects for 12 weeks. Since the porosity of the bioactive glass scaffolds was 50%, the amount of new bone estimated as a fraction of the pore area was about 17%. The results indicate that bioactive glass scaffolds with the grid-like microstructure had a microstructure more conductive to supporting bone ingrowth when compared to the oriented, trabecular, and fibrous microstructures. The amount of new bone formed after 6 weeks in vivo was approximately the same or greater than that in the oriented, trabecular, and fibrous microstructures implanted for 12 weeks.

### Example 7

### Histologic Processing

The calvarial samples, including the surgical sites with surrounding bone and tissue, were fixed in 10% buffered formaldehyde for 3 days, then transferred into 70% ethyl alcohol, and cut in half. Half of each sample was for paraffin embedding and the other half for methyl methacrylate embedding. The samples for paraffin embedding were de-siliconized by immersion for 2 h in 10% hydrofluoric acid, decalcified in 14% ethylenediaminetetraacetic acid (EDTA) solution for 4 weeks, dehydrated in a series of graded ethanol, and embedded in paraffin using routine histological techniques. Then the specimens were sectioned to 5 μm and stained with hematoxylin and eosin (H&E). The undecalcified samples were dehydrated in ethanol and embedded in PMMA. Sections were affixed to acrylic slides and ground down to 40 μm using a surface grinder (EXAKT 400CS, Norderstedt, Germany), and stained using the von Kossa technique. Transmitted light images of the stained sections were taken with an Olympus BX 50 microscope connected with a CCD camera (DP70, Olympus, Japan).

FIGS. 5A to 5E show von Kossa stained sections of the bioactive glass scaffolds and surrounding bone after the six-week implantation. The dark stain in the von Kossa technique is sensitive to the presence of phosphate (such as calcium phosphate in bone and in the modified surface layer of the bioactive glass scaffold). The rat calvarial defects implanted with the as-fabricated scaffolds showed little new bone formation (FIG. 5A); furthermore, the limited new bones formed were located in islands within the bioactive glass scaffolds. In comparison, the calvarial defects implanted with the surface modified bioactive glass scaffolds (no BMP-2) were almost completely filled with new bone (FIG. 5B). These results show that the bioactive glass scaffolds with a modified surface layer regenerate bone much faster than the as-fabricated scaffolds. Comparison of the results with published results for other studies showed that the 13-93 bioactive glass scaffolds with a modified surface layer regenerated bone far faster than other bioactive glass scaffolds, such as silicate, borate and borosilicate bioactive glass scaffolds with a fibrous microstructure.

FIGS. 5C-5E show von Kossa stained images of the bioactive glass scaffolds with a modified surface layer loaded with BMP-2. New bone (dark stain) was observed throughout the bioactive glass scaffold, but the amount of mature bone tissue was smaller than that for the surface modified bioactive glass scaffold without BMP-2. However, the amount of new bone in the bioactive glass scaffolds with BMP-2 was still higher and more evenly distributed than in bioactive glass scaffolds in other studies.

### Example 8

### Histomophometric Analysis

Histomorphometric analysis was carried out using optical images of the stained sections and Image J software. The percent new bone formed in the defects was evaluated from the H&E stained sections. The entire defect area was determined as the area between the two defect margins, including the entire bioactive glass scaffold and the tissue within. The available pore area within the bioactive glass scaffold was determined by subtracting the area of the bioactive glass scaffold from the total defect area. The newly formed bone, fibrous tissue, and bone marrow-like tissue within the defect area were then outlined and measured. The area of each tissue was expressed as a percentage of the total defect area as well as a percentage of the available pore area within the bioactive glass scaffold.

### Example 9

### Scanning Electron Microscopy

Unstained sections of the bioactive glass scaffolds in PMMA were coated with carbon and examined using a field-emission scanning electron microscope (SEM) (S-4700; Hitachi, Tokyo, Japan) operating in the backscattered electron (BSE) mode. The specimens were examined at an accelerating voltage of 15 kV and a working distance of 12 mm.

### Example 10

### Characteristics of Bioactive Glass Scaffolds and Modified Surface Layer

As fabricated, the bioactive glass scaffolds implanted in the rat calvarial defects had a grid-like microstructure (FIG. 1A), composed of almost fully dense bioactive glass filaments of diameter 330 □ 10 μm and pores of width 300±10 μm in the plane of deposition (xy plane) and 150±10 μm in the direction perpendicular to the deposition plane (z direction) (FIG. 1B). The porosity of the bioactive glass scaffolds, as measured using the Archimedes method, was 47±1%.

After reaction of the bioactive glass scaffolds in the K₂HPO₄ solution, the smooth dense surface of the glass filaments (FIG. 2A) became rough and porous (FIG. 2B), with a fine particulate morphology that was dependent on the reaction time. After reaction for 1 day, the modified surface layer appeared to consist mainly of nearly spherical nanoparticles with some fine needle-like particles (FIG. 3A). With an increase in the reaction time to 3 days, the amount of needle-like particles appeared to increase (FIG. 3B), whereas after reaction for 6 days, the surface consisted predominantly of needle-like particles (FIG. 3C). The cross-section of the glass filaments in the bioactive glass scaffolds showed the thickness of the modified surface layer (FIG. 2D). As the reaction time increased from 1 day to 6 days, the thickness of the converted layer increased from 2 μm to 13 μm, while the surface area increased from 19 m²/g to 47 m²/g (Table I).

FIG. 4 shows X-ray diffraction patterns of the scaffolds as-fabricated and after the surface modification in the aqueous phosphate solution for 1, 3, and 6 days. As fabricated, the scaffolds showed no measureable peaks; instead a broad band (centered at about 30° 2θ), typical of an amorphous material, was observed. After treatment for 1 day, small peaks corresponding to those of hydroxyapatite (the mineral constituent of bone) were observed, which increased in intensity with increase in the reaction time to 6 days. The XRD patterns confirm the formation of a calcium phosphate modified surface layer of the bioactive glass as a result of the reaction in the aqueous phosphate solution, which gradually converted to a hydroxyapatite-like material.

The XRD pattern of the converted layer formed after the one-day reaction also showed a broad bump at about 22° 28 in the vicinity of the major peak for the cristobalite phase of silica. The height of the bump gradually weakened with increasing reaction time. A similar bump has been observed in the XRD pattern of silicate 45S5 and 13-93 bioactive glass, and it has been attributed to the polymerization of silanol groups during the early stage of the conversion process, leading to the formation of a silica gel phase.

### Example 11

### Release Profile of BMP-2 from the Bioactive Glass Scaffolds In Vitro

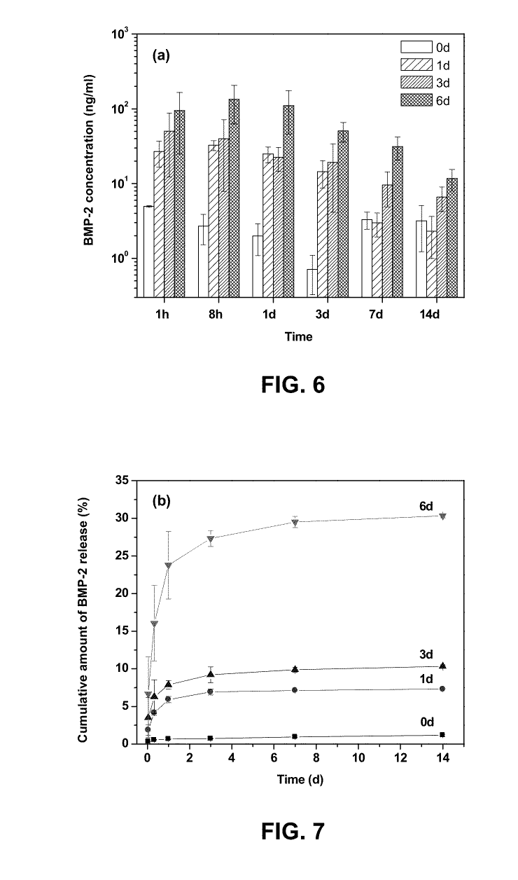

FIG. 6 shows the concentration of BMP-2 released into the medium at each time point. The data were used to determine the cumulative amount of BMP-2 released into the medium as a function of time (FIG. 7). The BMP-2 release profile from the pretreated bioactive glass scaffolds showed the same trend; a rapid burst release during day 1 was followed by a much slower release rate. However, at a given time, the amount of BMP-2 released into the medium increased with the duration of the pretreatment time (1-6 days) in the phosphate solution. In comparison, there was little release from the as-fabricated scaffolds (no surface treatment) that were loaded with BMP-2. After 14 days, about 30% of total BMP-2 initially loaded into the bioactive glass scaffolds was released from bioactive glass scaffolds pretreated for 6 days, which was significantly higher than the amount released from the scaffolds pretreated for 3 days (about 10%) or for 1 day (about 7%). In comparison, the amount of BMP-2 released from the as-fabricated scaffolds after the fourteen-day period was only about 1%. With an increase in pretreatment time from 1 to 6 days, both the thickness and the specific surface area of the converted layer increased (Table I). However, the phase composition and crystallinity of the converted layer also changed with the pretreatment time which could also influence the adsorption and release of the BMP-2.

### Example 12

### Assessment of Bone Regeneration and Integration

The results of this example show that the characteristics of the modified surface layer can have a marked effect on the capacity of the pretreated 13-93 bioactive glass scaffolds to enhance bone formation. H&E and von Kossa stained sections of bioactive glass scaffolds composed of the as-fabricated scaffolds and the bioactive glass scaffolds pretreated in the K₂HPO₄ solution for 1 day, 3 days, and 6 days which were implanted for 6 weeks in rat calvarial defects are shown in FIG. 8. The von Kossa positive area (dark stain) within the defect showed the presence of mineralized bone as well as hydroxyapatite (or calcium phosphate material) resulting from the pretreatment of the bioactive glass scaffolds or conversion in vivo. Because of the limited amount of hydroxyapatite formed in the pretreatment process and the conversion in vivo, the von Kossa positive area corresponded generally to the H&E stained areas.

All bioactive glass scaffolds showed the formation of new bone into the edges (periphery) of the bioactive glass scaffolds (adjacent to the old bone), indicating good integration of the bioactive glass scaffolds with the surrounding bone. New bone formation was observed mainly within the pores of the bioactive glass scaffolds, and the amount of new bone formed was dependent on the pretreatment in the aqueous phosphate solution. Scaffolds composed of the as-fabricated scaffolds showed a limited amount of new bone within the pores of the scaffolds, predominantly in the form of “islands” (FIGS. 8A1-A3). In comparison, the pretreated bioactive glass scaffolds showed a greater capacity to support new bone formation (FIGS. 8B1-D3). In particular, the bioactive glass scaffolds pretreated for 3 days in the K₂HPO₄ solution showed the greatest capacity to support new bone formation; the bioactive glass scaffold was well integrated with the old bone and the pores of the scaffold was almost completely infiltrated with new bone (FIG. 8C2). Blood vessels were observed within all of the implanted bioactive glass scaffolds in the defects (FIGS. 8A3-D3).

Infiltration of new bone into the grid-like bioactive glass scaffolds was also different in nature when compared to bioactive glass scaffolds of silicate 13-93 and borate-based bioactive glasses with the oriented and fibrous microstructures implanted in the same animal model. New bone infiltrated the grid-like bioactive glass scaffolds mainly from the edge (adjacent to old bone), indicating that new bone formation was mainly osteoconductive in nature, but some “islands” of new bone were also observed within the interior pores of the bioactive glass scaffold (FIG. 8A2). In comparison, while bone formation in the oriented and fibrous bioactive glass scaffolds was mainly osteoconductive, new bone formed mainly on the dural side of the bioactive glass scaffold with little infiltration into the edge for implantation times of up to 12 weeks. In the case of 13-93 bioactive glass scaffolds with the trabecular microstructure, new bone formation was found predominantly at the periphery of the defect. Differences in the nature of the new bone infiltration (from the edge vs. along the dural side of the bioactive glass scaffold) appear to be dependent on the size and interconnectivity of the pores. Larger pores with better pore interconnectivity appear to support greater bone infiltration from the edge of the bioactive glass scaffold.

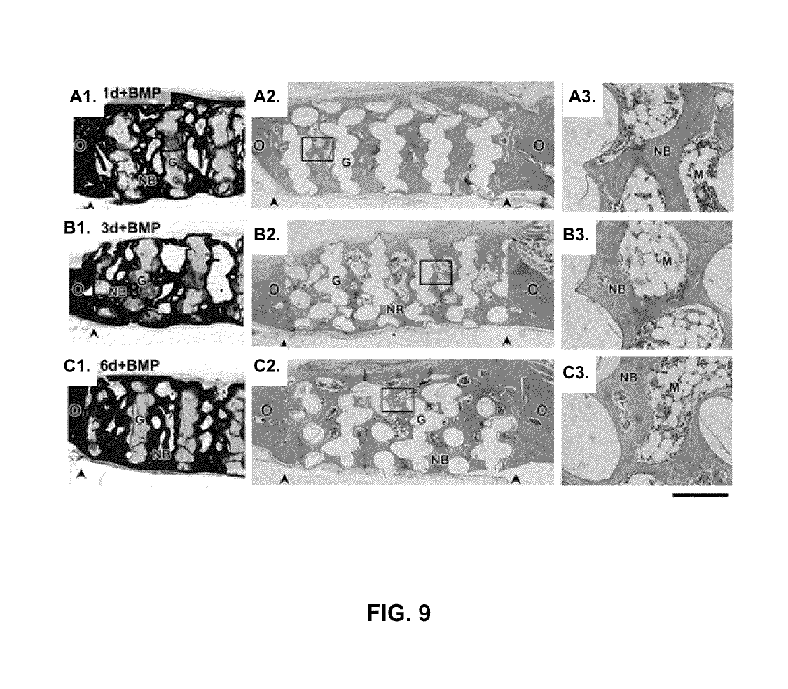

FIG. 9 shows H&E and von Kossa stained sections of the bioactive glass scaffolds composed of scaffolds that were pretreated for 1 day, 3 days, and 6 days and loaded with BMP-2 (1 μg per defect) after implantation for 6 weeks in rat calvarial defects. A considerable amount of new bone infiltrated the bioactive glass scaffolds and completely bridged the interface with old bone. When compared to the pretreated bioactive glass scaffolds described above (no BMP-2), these BMP-loaded bioactive glass scaffolds showed a markedly larger amount of bone marrow-like tissue that was surrounded by new bone within the pore space of the bioactive glass scaffolds (FIGS. 9A3-C3).

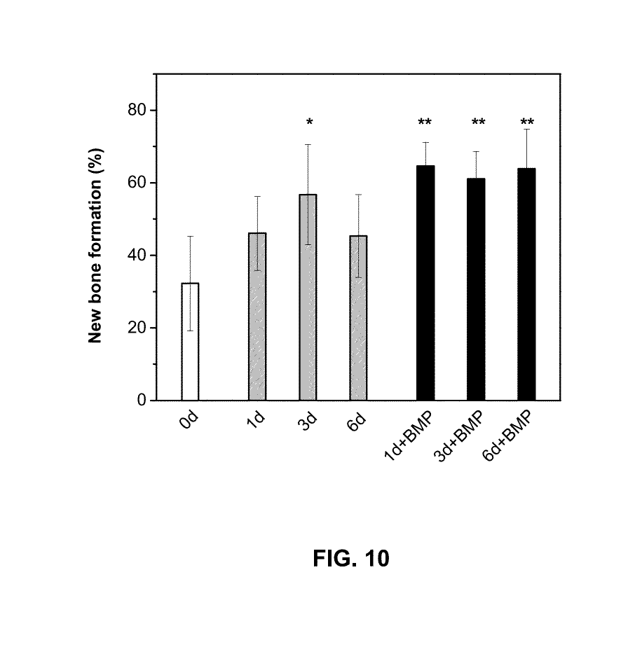

Since all the bioactive glass scaffolds had the same overall microstructure, the capacity of the bioactive glass scaffolds to regenerate bone was compared on the basis of new bone formed as a percent of the pore area of the bioactive glass scaffolds (FIG. 10, Table IV). Table IV shows the amount of new bone, fibrous tissue, and bone marrow-like tissue formed in bioactive glass (13-93) scaffolds implanted for 6 weeks in rat calvarial defects. (The amount of each tissue is expressed as a percent of the available pore space and total defect area in the bioactive glass scaffolds).

TABLE IV

| | | | | | Bone marrow- |
|---|---|---|---|---|---|
| | New bone (%) | | Fibrous tissue (%) | | like tissue (%) |
| | Available | Total | Available | Total | Available |
| Scaffold group | area | area | area | area | area |
| As fabricated (0 d) | 32 ± 13 | 18 ± 8 | 62 ± 14 | 34 ± 8 | 1 ± 1 |
| 1 d | 46 ± 10 | 25 ± 5 | 45 ± 12 | 25 ± 7 | 2 ± 1 |
| 3 d | 57 ± 14 | 33 ± 10 | 35 ± 13 | 19 ± 7 | 3 ± 2 |
| 6 d | 45 ± 11 | 26 ± 8 | 48 ± 13 | 28 ± 8 | 2 ± 1 |
| 1 d + BMP | 65 ± 7 | 38 ± 4 | 14 ± 12 | 8 ± 7 | 13 ± 6 |
| 3 d + BMP | 61 ± 8 | 35 ± 3 | 7 ± 7 | 4 ± 4 | 22 ± 8 |
| 6 d + BMP | 64 ± 11 | 38 ± 6 | 15 ± 19 | 10 ± 12 | 16 ± 8 |

The amount of new bone formed in bioactive glass scaffolds composed of the as-fabricated scaffolds after the six-week implantation was 32±13%. In comparison, the percent new bone formed in the bioactive glass scaffolds pretreated in K₂HPO₄ solution for 1 day, 3 days, and 6 days was 46±10%, 57±14%, and 45±11%, respectively. The bioactive glass scaffold pretreated for 3 days had a significantly higher percent of new bone growth than the as-fabricated scaffold (p<0.05). The amount of new bone formed in the bioactive glass scaffolds pretreated for 1 day, 3 days, and 6 days and loaded with BMP-2 was 65±7%, 61±8%, and 64±11%, respectively; these values were significantly higher than the percent new bone formed in the as-fabricated scaffolds and the bioactive glass scaffolds pretreated for 1 and 6 days.

The in vivo results show that when loaded with BMP-2 (1 μg/defect), all three bioactive glass scaffolds, pretreated for 1 day, 3 days, and 6 days in K₂HPO₄ solution, significantly enhanced bone regeneration when compared to the as-fabricated scaffold. The capacity of the BMP-loaded bioactive glass scaffolds to enhance bone regeneration after the six-week implantation was independent of the pretreatment time (FIG. 10). As described above, the release profile of BMP-2 from the bioactive glass scaffolds in vitro showed a large dependence on the pretreatment time. While the release of BMP-2 from the bioactive glass scaffolds in vivo is expected to be different from that in vitro, the results indicate that the amount of BMP-2 released from all three pretreated bioactive glass scaffolds might be adequate to stimulate bone regeneration. The larger amount of BMP-2 released from the bioactive glass scaffolds pretreated for 6 days, as observed from the in vitro release profile, may be more than the minimum amount required to stimulate bone formation.

When compared to the bioactive glass scaffolds subjected to the pretreatment alone (no BMP-2), the amount of new bone formed in the BMP-loaded bioactive glass scaffolds (61-65%) was not significantly greater than the value (57%) for the bioactive glass scaffolds pretreated for 3 days. In comparison, for pretreatment times of 1 day and 6 days, the BMP-loaded bioactive glass scaffolds showed a significantly greater amount of new bone formation when compared to the pretreated bioactive glass scaffolds (no BMP-2). Presumably because the three-day pretreatment alone was very effective, there was little opportunity for the beneficial effect of the BMP-2 to be felt.

While the capacity of the BMP-loaded bioactive glass scaffolds to regenerate bone and integrate with old bone was similar to that of the bioactive glass scaffolds pretreated for 3 days (no BMP-2), there were differences in the quality of the new bone. Apart from the new bone that infiltrated the pores of the bioactive glass scaffolds, the remaining pore space in the pretreated bioactive glass scaffolds was filled with soft tissue whereas, in the BMP-loaded bioactive glass scaffolds, the remaining pore space was filled with bone marrow-like tissue (FIG. 10). Formation of bone marrow-like tissue has also been reported in other biomaterials (such as hydrogel or calcium phosphate/hydrogel composites) loaded with BMP-2 after implantation for 2-8 weeks in an osseous defect, whereas the carrier alone showed far lower amounts of bone marrow-like tissue. It is likely that the mechanisms of bone regeneration in the bioactive glass scaffolds subjected to the pretreatment alone are different from those in the BMP-loaded bioactive glass scaffolds, but the details are currently unclear.

### Example 13

### Assessment of Bone Marrow and Soft Tissue Formation

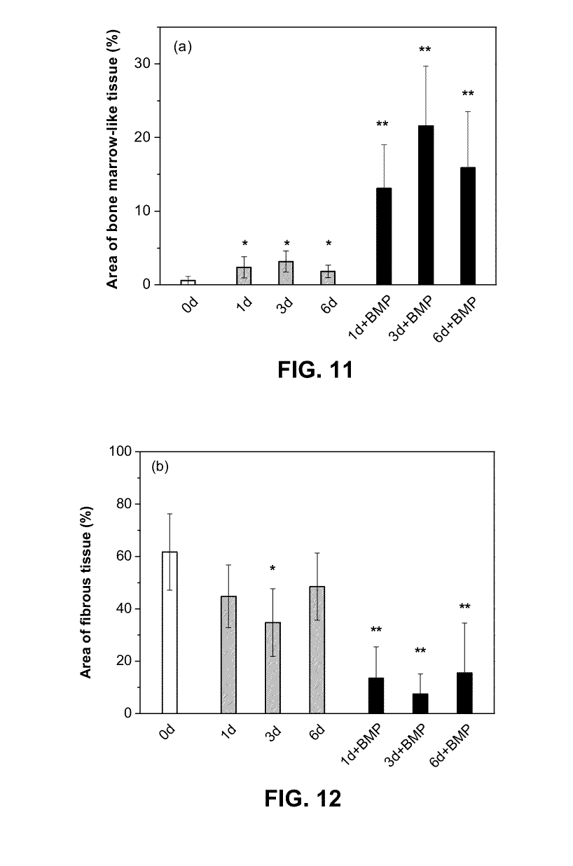

FIG. 11 shows that the amount of bone marrow-like tissue formed in the bioactive glass scaffolds composed of the pretreated bioactive glass scaffolds loaded with BMP-2 was higher than that in the bioactive glass scaffolds without BMP-2 (as-fabricated or pretreated bioactive glass scaffolds), but the amount of soft (fibrous) tissue was significantly lower. An interesting observation is that while the amount of new bone formed in the bioactive glass scaffolds pretreated for 3 days (57%) was not significantly different from that in the BMP-loaded bioactive glass scaffolds (61-65%), the type of tissue in the remainder of the pore space was very different. The remainder of the pore space in the pretreated bioactive glass scaffolds was filled mainly with fibrous tissue whereas the remaining pore space in the BMP-loaded bioactive glass scaffolds was filled mainly with bone marrow-like tissue.

FIG. 12 shows the percent of fibrous tissue formed in rat calvarial defects implanted with scaffolds of 13-93 glass at 6 weeks post implantation. FIG. 12 illustrates the amount of fibrous tissue formed in as-fabricated scaffolds (0 d); pretreated bioactive glass scaffolds for 1 day, 3 days, and 6 days in aqueous phosphate solution; and pretreated bioactive glass scaffolds loaded with BMP-2 (1 μg/defect).

### Example 14

### SEM Evaluation of Bioactive Glass Scaffolds

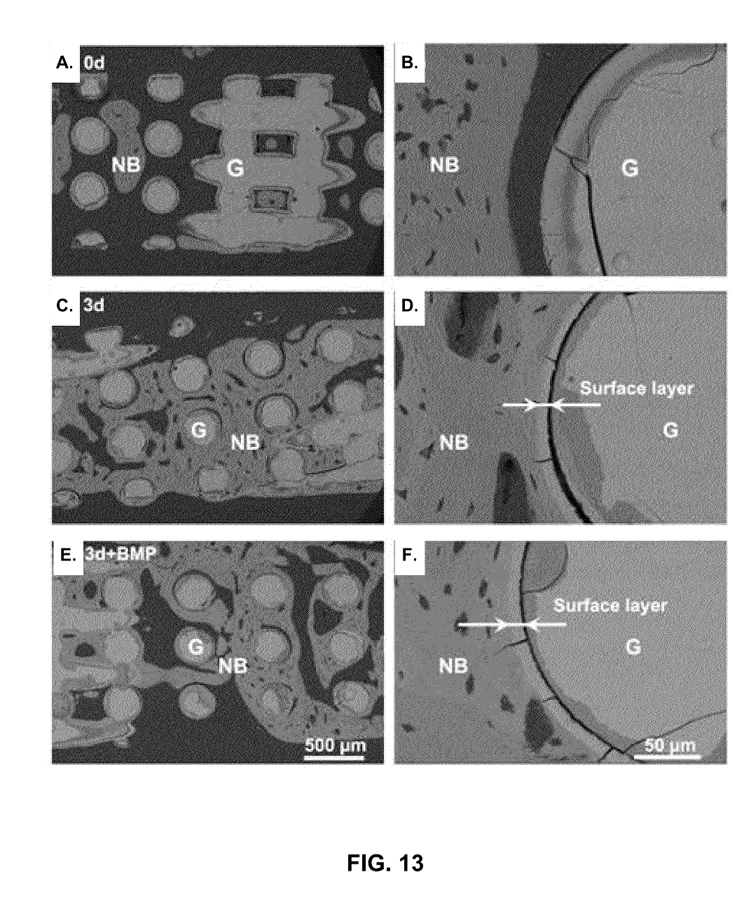

FIGS. 13A-13F show back-scattered SEM images of the cross-sections of bioactive glass scaffolds after the six-week implantation in rat calvarial defects. The contrast in the gray-scale images reveals differences in calcium content. The unconverted glass, HA-like material resulting from pretreatment of the bioactive glass scaffolds in the K₂HPO₄ solution prior to implantation and from conversion of the glass in vivo, and bone tissue, all with a high calcium content, had a light-gray color, while the silica-rich layer formed in the early stage of the conversion of the glass was dark-gray. In comparison, lacunae within the bone, fibrous tissue, and bone marrow-like tissue were almost black.

The glass filaments of the bioactive glass scaffolds consisted of three regions after implantation: an unconverted glass core (light-gray), a silica-rich layer (dark-gray) on the unconverted glass, and an HA-like modified surface layer (light-gray). (The cracks in the bioactive glass scaffolds and delamination of the HA layer presumably resulted from capillary drying stresses during the sample preparation for SEM examination.)

The bioactive glass scaffolds are composed of the as-fabricated scaffolds (FIGS. 13A and 13B) and the surface modified bioactive glass scaffolds without or with BMP-2 (FIGS. 13C-13F). These images confirm the far greater capacity of the surface modified bioactive glass scaffolds (without or with BMP-2) to regenerate bone. Furthermore, bioactive glass scaffolds show good bonding between the new bone and the modified surface layer of the bioactive glass scaffold. In comparison, the as-fabricated scaffolds show a gap between the new bone and the scaffold surface.

New bone formed during the six-week implantation did not appear to bond to the as-fabricated scaffolds (FIGS. 13A and B). Instead, the new bone formed islands within the pores of the bioactive glass scaffolds and there were large gaps between the newly formed bone and the surface of the bioactive glass scaffold. In comparison, new bone appeared to bond firmly to the modified surface layer of the pretreated scaffolds and the pretreated bioactive glass scaffolds loaded with BMP-2 (FIGS. 13C-F). The firm bonding to the modified surface layer of the bioactive glass scaffolds and the BMP-loaded scaffolds was found for all three pretreatment times (1 day, 3 days, and 6 days), but the images for the bioactive glass scaffolds pretreated for 1 day and 6 days are not included for the sake of brevity.

For the bioactive glass scaffolds pretreated for 3 days in the K₂HPO₄ solution prior to implantation (without or with BMP-2) (FIGS. 13D and F), the thickness of the HA layer (about 6 μm) after the six-week implantation was almost similar to the thickness (about 5 μm) prior to implantation (Table I). In comparison, the thickness of the HA layer formed on the as-fabricated scaffold after the six-week implantation was (about 20 μm), indicating that conversion of the as-fabricated scaffold in vivo was faster than that for the pretreated bioactive glass scaffold.

While the invention has been described in connection with specific embodiments thereof, it will be understood that the inventive device is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth.

What is claimed is:

1. A bioactive glass scaffold for new bone formation, the bioactive glass scaffold comprising:
a bioactive glass in a three-dimensional macroporous grid-like microstructure; and
a modified surface layer on the surface of the bioactive glass,
wherein the modified surface layer increases the surface area of the bioactive glass, and
wherein at least part of the modified surface layer comprises calcium phosphate.

2. The bioactive glass scaffold of claim 1, wherein at least part of the modified surface layer comprises a hydroxyapatite-like material.

3. The bioactive glass scaffold of claim 1 further comprising:
a plurality of biomolecules loaded on the modified surface layer, wherein the plurality of biomolecules comprises at least one of growth factors, antibodies, antibiotics, and drugs.

4. The bioactive glass scaffold of claim 3, wherein the plurality of biomolecules is a plurality of BMP-2.

5. The bioactive glass scaffold of claim 1, wherein the bioactive glass comprises at least one of a silicate, borate, phosphate, and borosilicate.

6. The bioactive glass scaffold of claim 5, wherein the bioactive glass is 13-93 silicate.

7. The bioactive glass scaffold of claim 1, wherein the bioactive glass scaffold has a porosity of about 50%.

8. The bioactive glass scaffold of claim 1, wherein the macropores of the bioactive glass scaffold are about 100 μm to about 500 μm in diameter.

9. A method of manufacturing a bioactive glass scaffold comprising:
grinding a bioactive glass into fine particles;
mixing the fine particles of bioactive glass with a processing aid and a liquid to form a slurry;
fabricating a three-dimensional macroporous grid-like microstructure of the slurry to form a bioactive glass scaffold; and
modifying the surface of the three-dimensional macroporous grid-like structure of the bioactive glass scaffold with a glass modifier,
wherein the modified surface layer increases the surface area of the bioactive glass scaffold to enhance new bone formation, and
wherein at least part of the modified surface layer comprises calcium phosphate.

10. The method of claim 9, wherein the fabrication step further comprises a thermal process to heat the bioactive glass scaffold after the fabricating step to dry the bioactive glass scaffold.

11. The method of claim 10, wherein the thermal process further comprises heating the bioactive glass scaffold to about 600° C. in flowing O₂ gas to decompose the processing aid.

12. The method of claim 10, wherein the thermal process further comprises heating the bioactive glass scaffold to about 700° C. in air to densify the bioactive glass scaffold.

13. The method of claim 10, wherein the thermal process further comprises heating the bioactive glass scaffold to about 250° C. to sterilize the bioactive glass scaffold.

14. The method of claim 13, wherein the thermal process lasts from about 1 hour to about 37 hours.

15. The method of claim 9 further comprising loading a plurality of biomolecules on the modified surface layer, wherein the plurality of biomolecules comprises at least one of growth factors, antibodies, antibiotics, and drugs.

16. The method of claim 15, wherein the plurality of biomolecules is a plurality of BMP-2.

17. The method of claim 9, wherein fabricating the macroporous grid-like microstructure comprises rapid prototyping or robotic deposition.

18. The method of claim 9, wherein modifying the surface of the bioactive glass comprises crystallization of the modified surface layer to hydroxyapatite.

19. The method of claim 9, wherein the glass modifier is selected from the group consisting of an alkali metal, an alkali-earth metal, or aqueous phosphate.

20. The method of claim 9, wherein modifying the surface of the bioactive glass scaffold with the glass modifier lasts from about 0.5 days to about 10 days.
